(19) United States
(12) Patent Application Publication
(10) Pub. No.: US 2021/0102162 A1
(43) Pub. Date: Apr. 8, 2021

(54) HARVESTING SYSTEMS AND METHODS FOR REMOVAL OF ALGAE SLURRY IMPURITIES

(71) Applicant: ExxonMobil Research and Engineering Company, Annandale, NJ (US)

(72) Inventors: Mark A. Deimund, Jersey City, NJ (US); Louis R. Brown, La Jolla, CA (US); Patrick L. Hanks, Bridgewater, NJ (US)

(21) Appl. No.: 17/019,422

(22) Filed: Sep. 14, 2020

Related U.S. Application Data
(60) Provisional application No. 62/912,263, filed on Oct. 8, 2019.

(51) Int. Cl. C12N 1/12; C12M 1/26

(52) U.S. Cl. CPC C12N 1/12; C12M 33/14

(57) ABSTRACT

A method includes containing an algae slurry within a cultivation vessel, introducing the algae slurry into a dissolved air floatation (DAF) system, and operating the DAF system in a low-recovery mode and thereby selectively removing impurities from the algae slurry. Algal biomass is then harvested from the algae slurry after removing the impurities from the algae slurry.

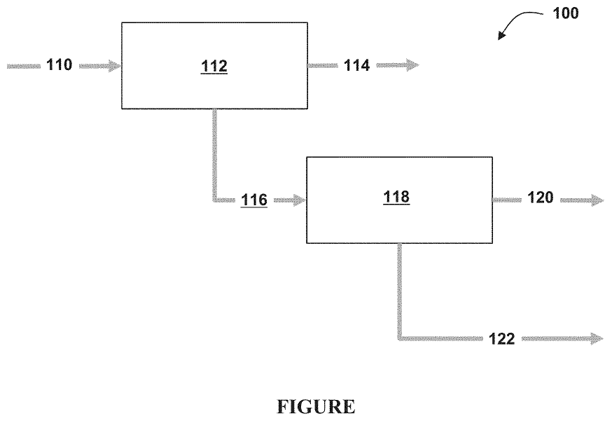

### BACKGROUND OF THE INVENTION

This application claims the benefit of priority from U.S. Provisional Application No. 62/912,263 filed Oct. 8, 2019, which is herein incorporated by reference in its entirety.

### FIELD OF THE INVENTION

The present disclosure relates to algae harvesting and, more particularly, to integration of an initial dissolved air floatation operation for removal of algae slurry impurities, and systems and methods related thereto.

### BACKGROUND OF THE INVENTION

Concerns about climate change, carbon dioxide (CO₂) emissions, and depleting mineral oil and gas resources have led to widespread interest in the production of biofuels from algae, including microalgae. As compared to other plant-based feedstocks, algae have higher CO₂ fixation efficiencies and growth rates, and growing algae can efficiently utilize wastewater, biomass residue, and industrial gases as nutrient sources.

Algae are photoautotrophic organisms that can survive, grow, and reproduce with energy derived entirely from the sun through the process of photosynthesis. Photosynthesis is essentially a carbon recycling process through which inorganic CO₂ combines with solar energy, other nutrients, and cellular biochemical processes to output gaseous oxygen and to synthesize carbohydrates and other compounds critical to the life of the algae.

To produce algal biomass, algae cells are generally grown in a water slurry comprising water and nutrients. The algae may be cultivated in indoor or outdoor environments, and in closed or open cultivation systems. Closed cultivation systems include photobioreactors, which utilize natural or artificial light to grow algae in an environment that is generally isolated from the external atmosphere. Such photobioreactors may be in a variety of shaped configurations, but are typically tubular or flat paneled. Open cultivation systems include natural and artificial ponds that utilize sunlight to facilitate photosynthesis. Artificial ponds are generally more preferred for industrial, scaled-up cultivation and are often shaped in circular (oval) or raceway-shaped configurations.

Various processing methods exist for harvesting cultivated algal biomass to extract lipids therefrom for the production of fuel and other oil-based products. Moreover, harvesting cultivated algal biomass can be used to produce non-fuel or non-oil-based products, including nutraceuticals, pharmaceuticals, cosmetics, chemicals (e.g., paints, dyes, and colorants), fertilizer and animal feed, and the like. Such methods traditionally include the addition of chemicals or the use of mechanical equipment to physically separate algae from the remaining components of a water slurry. However, various impurities may be present or otherwise introduced to the slurry during biomass cultivation. Traditional harvesting methods typically do not account for the presence and removal of such impurities, thereby maintaining the impurities during downstream processing and/or product production. In certain instances, the presence of such impurities may interfere with these processing and/or product production methods, including the quality of the resultant product itself.

Because algal biomass produces valuable commodities, including sustainable biofuels and non-oil based products, removal of impurities that may affect the quality and/or quantity of downstream processing methods and resultant products is desirable.

### SUMMARY OF THE INVENTION

In some embodiments, a method is disclosed and includes containing an algae slurry within a cultivation vessel, introducing the algae slurry into a dissolved air floatation (DAF) system, operating the DAF system in a low-recovery mode and thereby selectively removing impurities from the algae slurry, and harvesting algal biomass from the algae slurry after removing the impurities from the algae slurry.

In one or more embodiments, an algal biomass harvesting system is disclosed and may include a cultivation vessel configured contain an algae slurry for cultivation, a dissolved air floatation (DAF) system in fluid communication with the cultivation vessel and operable in a low-recovery mode to selectively remove impurities from the algae slurry, and an algae harvesting unit in fluid communication with the DAF system to harvest algal biomass from the algae slurry after removing the impurities from the algae slurry.

### BRIEF DESCRIPTION OF THE DRAWINGS

The FIGURE is included to illustrate certain aspects of the present disclosure, and should not be viewed as exclusive embodiments. The subject matter disclosed is capable of considerable modifications, alterations, combinations, and equivalents in form and function, without departing from the scope of this disclosure.

The FIGURE is a schematic diagram of an example two-step algal biomass harvesting method, according to one or more embodiments of the present disclosure.

### DETAILED DESCRIPTION OF THE INVENTION

Biofuel production from cultivated algae slurries offers sustainable energy solutions to reduce reliance on fossil fuels and reduce greenhouse gas emissions. Other non-oil-based products can additionally be derived from algal biomass. To accomplish substantial economic, environmental, and societal impact, algae must be cultivated in large-scale systems. Such large-scale cultivation systems allow algae-derived fuels and other non-oil-based products to become more cost-effective and more widely available to the public. However, impurities may be introduced or otherwise flourish during the cultivation of an algae slurry, particularly in open cultivation systems. Such impurities may include, but are not limited to, ash, dirt, diatoms, other foreign material (e.g., non-algal matter), and combinations thereof. Indeed, algae cells are often cultivated in algae slurries that may be upwards of thousands of liters or more in volume, and the concentration of impurities within such large volumes can be quite substantial. Such impurities may interfere (e.g., toxic or reactive) with downstream processing operability and may reduce the quality of the final biomass product. For instance, certain impurities commonly found in algae slurries may react with lipid extraction methods, and may adversely affect cold temperature operability of produced biofuel.

Traditionally, impurities found within algae slurries are collected together with harvested biomass, and various aqueous or chemical methods may be employed to remove or otherwise reduce the concentration of the impurities. Traditional impurity removal methods include, for example, water or chemical washing or extraction. However, such methods may be accompanied by significant financial, operational, and energy costs. Further, in some instances, traditional impurity removal methods may employ chemistries that are harmful to operators and the environment, and thereby furthering costs associated with accidents and necessary disposal or cleanup.

The present disclosure provides mechanical, two-step algae harvesting systems and methods configured to more efficiently remove impurities and thereby harvest purer algal biomass that does not rely on costly, and often harmful, chemical methods. The first or initial step utilizes a dissolved air floatation (DAF) operation or system for the removal of impurities from an algae slurry. Thereafter, the second step includes the separation or bulk collection of algal biomass for further downstream processing. Accordingly, the embodiments of the present disclosure facilitate initial separation of undesirable impurities, such as those described above, from desirable algal biomass prior to separating and recovering the algal biomass, thereby, among other things, improving average lipid yield and resulting in overall cleaner downstream processing and resultant products. Moreover, as described in greater detail below, the embodiments of the present disclosure may advantageously further be utilized to selectively isolate more robust, lipid-rich algae from less robust, less lipid-rich algae (i.e., less healthy algae cells).

As used herein, the term “algae slurry” or “algae water slurry,” and grammatical variants thereof, refers to a flowable, liquid comprising at least water, algae cells, and algae nutrient media (e.g., phosphorous, nitrogen, and optionally additional elemental nutrients).

Algal sources for the preparing the algae slurry include, but are not limited to, unicellular and multicellular algae. Examples of such algae can include, but are not limited to, a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurochrysis, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species.

The water used in preparing the algae slurry may originate from any water source including, but not limited to, fresh water, brackish water, seawater, wastewater (treated or untreated), synthetic seawater, and any combination thereof.

The algae nutrient media for use in forming an algae slurry may comprise at least nitrogen (e.g., in the form of ammonium nitrate or ammonium urea) and phosphorous. Other elemental micronutrients may also be included, such as potassium, iron, manganese, copper, zinc, molybdenum, vanadium, boron, chloride, cobalt, silicon, and the like, and any combination thereof.

As used herein, “dissolved air floatation” or “DAF,” and grammatical variants thereof, refers to any means of injecting pressurized gas (e.g., air, CO₂, nitrogen, and the like) into an algae slurry to suspend (float) solid matter and impurities. In example operation of a DAF system, the injected pressurized gas forms bubbles that adhere to solid matter within the algae slurry, and causes the solid matter to suspend or otherwise float to the surface of the algae slurry. In some applications, chemicals (e.g., flocculants and coagulants) may be added to the algae water slurry to help facilitate separation and improve the suspension.

In some embodiments, DAF may be integrated with a cultivation vessel, such that it is in fluid communication with the cultivation vessel and forms an integral part thereof. For example, an air sparger or other equipment configured to introduce a gas may be utilized within or adjacent to a cultivation vessel containing the algae slurry. In integrated embodiments, the location(s) at which the pressurized gas is injected into a cultivation vessel (i.e., into an algae slurry) may further include a skimming device (e.g., a weir) configured to continuously remove impurities and less healthy algae cells during cultivation, thereby reducing contamination and improving the robustness of the resultant algal biomass. Algae “health” can be quantified by measuring photosynthetic activity, and cell size can be determined by cell-sizing equipment, such as microscopes or light-scattering devices. Moreover, in integrated embodiments, algal seed concentrations can be reduced, thereby improving algal facility production. Alternatively, the initial DAF step may be integrated with a cultivation vessel and used only at the end of a cultivation run (i.e., at the time that the algal biomass is ready for harvesting and no longer requires cultivation).

In other embodiments, however, the DAF may not be integrated (or integral) with the cultivation vessel, but may instead comprise a separate structure apart from the cultivation vessel but nonetheless in fluid communication therewith. In such embodiments, with the cultivated algae slurry may be pumped out of the cultivation vessel and transferred to the DAF unit (system) via suitable piping, plumbing, and valves for impurity removal.

As used herein, the term “cultivation vessel,” and grammatical variants thereof, refers to any open or closed algae cultivation system used for the growth of algal biomass, including bioreactors, photobioreactors, natural ponds, artificial ponds (e.g., raceway ponds), and the like. While some embodiments of the present disclosure are described with reference to open cultivation systems (i.e., natural or artificial ponds) having an integrated DAF in fluid communication therewith, it is to be appreciated that either open or closed cultivation systems may be used in accordance with the embodiments of the present disclosure.

The present disclosure provides systems and methods of two-step algal biomass harvesting, in which the first step is configured to remove undesirable impurities and the second step is configured to recover desirable algal biomass. In particular, the systems and methods utilize an initial DAF step to remove the undesirable impurities, followed by secondary harvesting of the algal biomass.

Referring now to the FIGURE, illustrated is a schematic diagram depicting an example two-step algal biomass harvesting method 100, in accordance with one or more embodiments of the present disclosure. As shown, a cultivated algae slurry 110 may be initially introduced into a dissolved air floatation (DAF) system 112. The algae slurry 110 may be cultivated within a cultivation vessel and, in some embodiments, the DAF system 112 may be integrated with the cultivation vessel. In other embodiments, however, the DAF system 112 may comprise a separate DAF unit in fluid communication with the cultivation vessel.

As the algae slurry 110 is treated in the DAF system 112, impurities 114 present within the algae slurry 110 are floated to the surface of the algae slurry 110 for removal. In some embodiments, the DAF system 112 may include or otherwise incorporate a skimmer or weir configured to receive and remove at least a portion of the impurities 114 from the algae slurry 110, thus converting (transitioning) the algae slurry 110 into a reduced-impurities algae slurry 116. It is to be noted that the term “reduced-impurities,” and grammatical variants thereof, refers to a cultivated algae slurry that has at least a portion, up to all, of impurities removed.

The reduced-impurities algae slurry 116 may subsequently be treated or circulated through an algae harvesting unit 118 configured to separate and remove cultivated algal biomass 120. The algae harvesting unit 118 may comprise any harvesting separation unit configured to separate the cultivated algal biomass 120 from the remaining portions of the reduced-impurities algae slurry 116. In some embodiments, for example, the algae harvesting unit 118 may comprise a second DAF unit with an associated skimmer or weir. In other embodiments, however, the algae harvesting unit 118 may comprise a membrane separator or the like. In yet other embodiments, the algae harvesting unit 118 may comprise a centrifuge separator. After removing the cultivated algal biomass 120 from the reduced-impurities algae slurry 116, clarified water 122 may remain and may be recycled or otherwise treated for reuse or environmental disposal.

Accordingly, as shown, one or more methods of the present disclosure may process a cultivated algae slurry stream 110 into three resultant product streams of (1) an impurity stream 114 (e.g., ash, diatoms, and the like), (2) a concentrated algal biomass stream 120, and (3) a clarified water stream 122. The concentrated algal biomass stream 120 may be utilized for further downstream processing, such as lipid extraction for the production of biofuel.

In some embodiments, the DAF system 112 may be operated in a low-recovery mode in order to selectively float the impurities in the cultivated algae slurry 110 to the surface. As used herein, the term “low-recovery mode,” and grammatical variants thereof, refers to reducing one or more parameters of the DAF system 112 that would typically be used for recovery of algae cell-sized solids. One example low-recovery mode includes reducing a gas sparging (flow) rate as compared to the gas sparge rate in a DAF system configured for algae cell-sized solids recovery mode. This may be monitored and quantified by reducing the gas sparging rate by a certain (known) percentage. Another example low-recovery mode includes reducing the flow rate of the algae slurry 110 through the DAF system 112 as compared to the flow rate through a DAF system configured for algae cell-sized solids recovery mode. This may be monitored and quantified by reducing the flow rate of the algae slurry 110 by a certain (known) percentage. Yet another example low-recovery mode includes reducing the amount of chemical loading (addition) used to separate the impurities 114 from the mature algae, as compared to the amount of chemical loading commonly used in a DAF system configured for algae cell-sized solids recovery. This may be monitored and quantified by reducing the chemicals addition by a certain (known) percentage.

The low-recovery mode(s) for the DAF system 112 surprisingly permits floatation or suspension of solids that are smaller in size compared to algae cells, which are characteristic of undesirable impurities. In some but not all cases, solid particles within a cultivated algal slurry may be represented by a bimodal particle size distribution, wherein the smaller solid particles are undesirable impurities and the larger solid particles are desirable, lipid-rich algae cells (algal biomass). Moreover, and advantageously, algae cells that are less lipid-rich or less healthy are also smaller in size compared to the desirable, lipid-rich algae cells, and thus may fall into the particle size distribution with the undesirable impurities. Accordingly, in some embodiments, operating the DAF system 112 in a low-recovery mode may advantageously suspend smaller celled impurities 114 and less healthy algae cells for initial removal from the cultivated algae slurry 110 in the “float” or “surface” layer of the slurry 110. In contrast, the desirable, lipid-rich algae cells (i.e., the reduced-impurities algae slurry 116) will generally remain in an “underflow” layer of the algae slurry 110 and otherwise below the surface layer of the slurry 110 that contains the floated impurities 114. Consequently, the lipid-rich algae cells remain in the reduced-impurities algae slurry 116 while the float layer of impurities 114 and less healthy algae cells are removed, such as by a skimmer. In some embodiments, the recovered float layer comprising the impurities 114 may be thereafter sent for disposal or treatment.

It is noted, however, that in other cases lipid-rich algae cells may be generally smaller for some strains of algae, and the impurities can be smaller also. Accordingly, in such cases the operating parameters of the low-recovery mode(s) for the DAF system 112 would have to be altered to ensure proper entrainment of the impurities as opposed to the desirable, lipid-rich algae cells (algal biomass).

In some embodiments, the algae slurry 110 may be continuously introduced into the DAF system 112 during cultivation of the algae in the algae slurry 110 and otherwise throughout the entire residence time of the algae slurry 110 in the cultivation vessel. As a result, the DAF system 112 may be operable as a continuous impurities 114 removal step in the system 100 configured to remove unwanted contamination and species, such as diatoms, which may improve the robustness of the algal growth culture and help improve algal facility productivity. In such embodiments, CO₂ may be sparged into the algae slurry 110 at the DAF system 112 to not only float impurities 114 to the surface but also feed the algae slurry 110. Alternatively, or in addition thereto, the DAF system 112 may be employed in the system 100 at the end-of-run; i.e. , when the algae slurry 110 is ready to be harvested. In such embodiments, air may be sparged into the algae slurry 110 at the DAF system 112 to float the impurities 114 to the surface.

In some embodiments, prior to introducing the algae slurry 110 to the low-recovery DAF system 112, the biomass harvesting method 100 may further include an initial filtration step configured to reduce the amount of non-algal material in downstream processes. The initial filtration step may comprise any type of filtration device or assembly employed prior to the low-recovery DAF system 112, thereby reducing the impurities 114 that the DAF system 112 must remove. In at least one embodiment, the initial filtration step may comprise sedimentation of the algae.

The second recovery step undertaken at the algae harvesting unit 118 may employ any type of algal biomass recovery system or method, such as floatation, filtration, or sedimentation, without departing from the scope of the present disclosure. For example, in some embodiments, the algae harvesting unit 118 may comprise a subsequent (second) DAF system operable in an algae cell-sized solids recovery mode. In other embodiments, the algae harvesting unit 118 may comprise a centrifuge separator or a membrane filter or separator. In yet other embodiments, the algae harvesting unit 118 may comprise a combination of the foregoing algal biomass recovery systems or methods.

The recovered algal biomass 120 using the two-step process 100 of the present disclosure is more pure as compared to traditionally recovered algal biomass because at least a portion, or all, of the impurities 114 and less healthy algae cells have been removed. After recovery, the algal biomass 120 may be further processed for production of desirable biofuels or products (e.g., downstream cell lysis and lipid extraction). Further, the now algae-free slurry water 122 may remain in the cultivation vessel for subsequent algae cultivation purposes without the need to re-transport (recirculate) water back to the cultivation vessel or the need to refill an entire new volume of water and nutrients for cultivation. In other embodiments, the now algae-free slurry water 122 may be treated and disposed in an environmentally friendly manner

To facilitate a better understanding of the embodiments described herein, the following comparative example is described. In no way should the following example be read to limit, or to define, the scope of the invention.

To qualify the effectiveness of the systems and methods described herein employing an initial low-recovery mode DAF step, a cultivated water slurry stream was processed at ExxonMobil and Synthetic Genomics, Inc.'s California Advanced Algal Facility. First, an initial low-recovery DAF step was utilized and the float layer thereof was collected for visual observation. The underflow layer was additionally collected for visual observation. The float layer was visually opaque with concentrated solid impurities, including ash and diatoms. Differently, the underflow layer was translucent and comprised visible, green algae cells. A portion of both collected layers was centrifuged and observed. The centrifuged float layer was characterized by brown discoloration from the impurities, whereas the centrifuged underflow layer was characterized by a deep green color representing purer, harvested algae.

### EMBODIMENTS LISTING

The present disclosure provides, among others, the following embodiments, each of which may be considered as optionally including any alternate embodiments.

Clause 1. A method includes containing an algae slurry within a cultivation vessel, introducing the algae slurry into a dissolved air floatation (DAF) system, operating the DAF system in a low-recovery mode and thereby selectively removing impurities from the algae slurry, and harvesting algal biomass from the algae slurry after removing the impurities from the algae slurry.

Clause 2. The method of Clause 1, wherein operating the DAF system in the low-recovery mode comprises reducing a gas sparge rate through the algae slurry.

Clause 3. The method of Clause 1, wherein operating the DAF system in the low-recovery mode comprises reducing a flow rate of the algae slurry through the DAF system.

Clause 4. The method of Clause 1, wherein operating the DAF system in the low-recovery mode comprises reducing an amount of chemical loading used to separate the impurities from algae within the algae slurry.

Clause 5. The method of any of the preceding Clauses, wherein introducing the algae slurry into the DAF system is preceded by filtering the algae slurry prior to selectively harvesting the impurities from the algae slurry.

Clause 6. The method of Clause 5, wherein filtering the algae slurry comprises sedimentation of the algal biomass.

Clause 7. The method of any of the preceding Clauses, wherein harvesting the algal biomass from the algae slurry comprises circulating the algae slurry through an algae harvesting unit selected from the group consisting of a second DAF system, a membrane separator, a centrifuge, and any combination thereof.

Clause 8. The method of any of the preceding Clauses, wherein the impurities include one or more of ash, dirt, diatoms, non-algal matter, and any combination thereof.

Clause 9. The method of any of the preceding Clauses 1, wherein the impurities exhibit a first particle size distribution and the algal biomass exhibits a second particle size distribution different than the first particle size distribution.

Clause 10. An algal biomass harvesting system includes a cultivation vessel configured contain an algae slurry for cultivation, a dissolved air floatation (DAF) system in fluid communication with the cultivation vessel and operable in a low-recovery mode to selectively remove impurities from the algae slurry, and an algae harvesting unit in fluid communication with the DAF system to harvest algal biomass from the algae slurry after removing the impurities from the algae slurry.

Clause 11. The system of Clause 10, wherein the DAF system forms an integral part of the cultivation vessel.

Clause 12. The system of Clause 10, wherein the DAF system comprises a separate structure apart from the cultivation vessel.

Clause 13. The system of any of Clauses 10 to 12, wherein the low-recovery mode comprises reducing a gas sparge rate through the algae slurry.

Clause 14. The system of any of Clauses 10 to 12, wherein the low-recovery mode comprises reducing a flow rate of the algae slurry through the DAF system.

Clause 15. The system of any of Clauses 10 to 12, wherein the low-recovery mode comprises reducing an amount of chemical loading used to separate the impurities from algae within the algae slurry.

Clause 16. The system of any of Clauses 10 to 15, wherein the impurities comprise one or more of ash, dirt, diatoms, non-algal matter, and any combination thereof.

Clause 17. The system of any of Clauses 10 to 16, wherein the impurities exhibit a first particle size distribution and the algal biomass exhibits a second particle size distribution different than the first particle size distribution.

Clause 18. The system of Clause 10, further comprising a filter that filters the algae slurry prior to being introduced into the DAF system.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The invention claimed is:

1. A method, comprising:
containing an algae slurry within a cultivation vessel;
introducing the algae slurry into a dissolved air floatation (DAF) system;
operating the DAF system in a low-recovery mode and thereby selectively removing impurities from the algae slurry; and
harvesting algal biomass from the algae slurry after removing the impurities from the algae slurry.

2. The method of claim 1, wherein operating the DAF system in the low-recovery mode comprises reducing a gas sparge rate through the algae slurry.

3. The method of claim 1, wherein operating the DAF system in the low-recovery mode comprises reducing a flow rate of the algae slurry through the DAF system.

4. The method of claim 1, wherein operating the DAF system in the low-recovery mode comprises reducing an amount of chemical loading used to separate the impurities from algae within the algae slurry.

5. The method of claim 1, wherein introducing the algae slurry into the DAF system is preceded by filtering the algae slurry prior to selectively harvesting the impurities from the algae slurry.

6. The method of claim 5, wherein filtering the algae slurry comprises sedimentation of the algal biomass.

7. The method of claim 1, wherein harvesting the algal biomass from the algae slurry comprises circulating the algae slurry through an algae harvesting unit selected from the group consisting of a second DAF system, a membrane separator, a centrifuge, and any combination thereof.

8. The method of claim 1, wherein the impurities include one or more of ash, dirt, diatoms, non-algal matter, and any combination thereof.

9. The method of claim 1, wherein the impurities exhibit a first particle size distribution and the algal biomass exhibits a second particle size distribution different than the first particle size distribution.

10. An algal biomass harvesting system, comprising:
a cultivation vessel configured contain an algae slurry for cultivation;
a dissolved air floatation (DAF) system in fluid communication with the cultivation vessel and operable in a low-recovery mode to selectively remove impurities from the algae slurry; and
an algae harvesting unit in fluid communication with the DAF system to harvest algal biomass from the algae slurry after removing the impurities from the algae slurry.

11. The system of claim 10, wherein the DAF system forms an integral part of the cultivation vessel.

12. The system of claim 10, wherein the DAF system comprises a separate structure apart from the cultivation vessel.

13. The system of claim 10, wherein the low-recovery mode comprises reducing a gas sparge rate through the algae slurry.

14. The system of claim 10, wherein the low-recovery mode comprises reducing a flow rate of the algae slurry through the DAF system.

15. The system of claim 10, wherein the low-recovery mode comprises reducing an amount of chemical loading used to separate the impurities from algae within the algae slurry.

16. The system of claim 10, wherein the impurities comprise one or more of ash, dirt, diatoms, non-algal matter, and any combination thereof.

17. The system of claim 10, wherein the impurities exhibit a first particle size distribution and the algal biomass exhibits a second particle size distribution different than the first particle size distribution.

18. The system of claim 10, further comprising a filter that filters the algae slurry prior to being introduced into the DAF system.
